(19) United States
(12) Patent Application Publication
(10) Pub. No.: US 2026/0048232 A1
(43) Pub. Date: Feb. 19, 2026

(54) CATHETER SHAFT AND METHODS OF MAKING A CATHETER SHAFT

(71) Applicant: Boston Scientific Scimed, Inc., Maple Grove, MN (US)

(72) Inventors: Emily Rose Whitwam, Osseo, MN (US); Jason John Matteson, Beldenville, WI (US); David Cory Kirt, Minnetonka, MN (US)

(21) Appl. No.: 19/301,281

(22) Filed: Aug. 15, 2025

Related U.S. Application Data
(60) Provisional application No. 63/684,101, filed on Aug. 16, 2024.

(51) Int. Cl. A61M 25/00; A61M 25/01

(52) U.S. Cl. CPC A61M 25/005; A61M 25/0012; A61M 25/0136

(57) ABSTRACT

A medical catheter and its manufacturing method are presented, creating a single device from two distinct components. The catheter features a proximal shaft and a distal shaft with distinct polymeric material where the exposed braided structures are mechanically connected at a transition portion, resulting in a single shaft. A reinforcing assembly is integrated into the proximal shaft and includes reinforcing sleeves with helical coils creating a steering wire guidance system, and an anchor ring mechanically connected to the transition portion. The distal shaft incorporates an articulation structure with interconnected elements and steering wire lumens aligned with the reinforcing assembly. The manufacturing process involves selectively removing polymeric jackets of a proximal and distal shaft, exposing the braided structures, and mechanically connecting the braided structures. Such a medical catheter device and method of manufacturing addresses limitations of conventional joining methods, enhancing maneuverability and precision for navigating complex anatomies in various applications.

### CROSS REFERENCE TO RELATED APPLICATIONS

This application claims priority to U.S. Provisional Patent Application No. 63/684,101 entitled “TWO PIECE R2R SHAFT ALL COMPONENTS WELDED,” filed Aug. 16, 2024, which is hereby incorporated by reference in its entirety.

### TECHNICAL FIELD

The present invention relates to medical devices and methods for catheters for medical procedures. More specifically, the invention relates to devices and methods that include structural and directional enhancements for catheters such as steerable catheters.

### BACKGROUND

Medical catheters incorporate flexible distal shafts for navigation as well as reinforced proximal shafts for pushability and torque response. However, joining proximal and distal catheter shafts of different materials and properties pose manufacturing challenges. Typical methods such as thermal bonding, adhesive bonding and reflowing proximal and distal shafts into an integrated device can be inconsistent and time intensive. In addition, the connection region between the joined proximal and distal shafts often exhibits abrupt mechanical property changes in stiffness and strength. These inefficiencies can impede positioning and performance capabilities of the catheter.

Therefore, there is a need for improved connection methods of integrating independent medical catheter shaft components into a single medical catheter device that enables greater catheter performance.

### SUMMARY

In Example 1, a medical catheter comprising a handle configured for manipulation by a user, and a flexible shaft having a proximal portion, a distal portion, and a transition portion between the proximal and distal portions of the shaft. The proximal portion of the shaft extends distally from the handle and is formed from a proximal shaft component having a proximal reinforcing braid and a proximal polymeric jacket disposed over the proximal reinforcing braid, wherein a first length of the proximal reinforcing braid extends distally of the proximal polymeric jacket. The distal portion of the shaft is formed from a distal shaft component having a distal reinforcing braid and a distal polymeric jacket disposed over the distal reinforcing braid, wherein a second length of the distal reinforcing braid extends proximally of the distal polymeric jacket and is mechanically connected to the first length of the proximal reinforcing braid. The transition portion of the shaft comprises the first length of the proximal braid member, the second length of the distal braid member, and a transition portion jacket disposed over the first length of the proximal braid member and the second length of the distal braid member.
In Example 2, the medical catheter of Example 1, wherein the proximal portion comprises a reinforcing assembly disposed within the proximal shaft component. The reinforcing assembly comprises a pair of diametrically opposed helical reinforcing sleeves, wherein the reinforcing sleeves establish reinforced passageways configured to provide lateral support for one or more steering elements extending from the proximal region to the distal region, and an anchor ring attached to a distal end of each of the reinforcing sleeves, the anchor ring comprising a ring structure and including passageways for the one or more steering elements, and wherein the anchor ring is mechanically connected to the proximal shaft component and the distal shaft component in the transition portion.
In Example 3, the medical catheter of Example 2, wherein the distal portion comprises a deflectable region defining a longitudinal axis and further comprising an articulation structure disposed within the distal shaft component. The articulation structure comprises a plurality of longitudinally-arranged tubular segments and a plurality of connecting segments connecting adjacent tubular segments, one or more steering wire lumens extending through the tubular segments and aligned with the reinforcing sleeves, first and second reinforcing members extending through the tubular segments and the connecting segments.
In Example 4, the medical catheter of Example 3, wherein the deflectable region is configured to assume a curved shape when a deflection force is applied to the distal portion of the shaft.
In Example 5, the medical catheter of Example 3, wherein the one or more steering wire lumens are each circumferentially offset from the first and second reinforcing members by about 90 degrees.
In Example 6, the medical catheter of Example 2, wherein the first length of the proximal reinforcing braid and the second portion of the distal reinforcing braid overlap one another in the transition portion.
In Example 7, the medical catheter of Example 2, comprising a mechanical connection connecting the first length of the proximal reinforcing braid and the second length of the distal reinforcing braid.
In Example 8, the medical catheter of Example 7, wherein the mechanical connection further connects the anchor ring to the first length of the proximal reinforcing braid and the second length of the distal reinforcing braid.
In Example 9, the medical catheter of Example 7, wherein the transition portion jacket is formed from polymeric material reflowed over and around the mechanical connection connecting the first length of the proximal reinforcing braid and the second length of the distal reinforcing braid.
In Example 10, the medical catheter of Example 9, wherein the mechanical connection is formed by at least one joining method including welding process, soldering process, adhesive process, polymer reflow process, or combination thereof.
In Example 11, the medical catheter of Example 8, wherein the anchor ring includes a groove defined in its outer circumference, and wherein the mechanical connection is formed by disposing an adhesive material over the first length of the proximal reinforcing braid and the second length of the distal reinforcing braid and into the groove.
In Example 12, the medical catheter of Example 8, wherein the mechanical connection comprises a weld joint connecting the first length of the proximal reinforcing braid, the second length of the distal reinforcing braid, and the anchor ring.
In Example 13, the medical catheter of any of Examples 1-12, wherein the proximal polymeric jacket and the distal polymeric jacket are made from different polymeric materials.
In Example 14, the medical catheter of any of Examples 1-13, wherein the first length of the proximal reinforcing braid and the second length of the distal reinforcing braid are dimensioned to define, in the absence of the transition portion jacket, a gap between the proximal polymeric jacket and the distal polymeric jacket having a length of about 0.030 inches to about 0.300 inches.
In Example 15, the medical catheter of Example 14, wherein the transition portion jacket fills and seals the gap.
In Example 16, a medical catheter comprising a handle configured for manipulation by a user, and an elongate shaft proximal portion, an elongate shaft distal portion, a reinforcing assembly and an articulation structure. The elongate proximal shaft portion extends from the handle and comprises a proximal reinforcing braid disposed within a proximal polymeric jacket, wherein a first length of the proximal reinforcing braid extends distally beyond the proximal polymeric jacket. The elongate distal shaft portion comprises a distal reinforcing braid disposed within a distal polymeric jacket, wherein a second length of the distal reinforcing braid extends proximally beyond the distal polymeric jacket, wherein the first length of proximal reinforcing braid and the second length of distal reinforcing braid are mechanically connected to form an integrated transition portion joining the proximal and distal shaft portions. The reinforcing assembly extends proximally from the transition portion within the proximal shaft portion, wherein the reinforcing assembly comprises a pair of diametrically opposed helical reinforcing sleeves, and an anchor ring attached to the distal end of the reinforcing sleeves, wherein the reinforcing sleeves establish passageways for one or more steering elements extending through the proximal shaft portion. The articulation structure extends distally from the transition portion, wherein the articulation structure comprises a plurality of longitudinally-arranged articulation elements, wherein the articulation elements comprise a plurality of tubular segments and a plurality of connecting segments connecting adjacent tubular segments, and one or more reinforcing members extending through the tubular segments and the connecting segments, the articulation structure configured to enable deflection of the distal shaft portion along a curved plane.
In Example 17, the medical catheter of Example 16, wherein the articulation structure is configured to assume a curved shape when a deflection force is applied to the distal shaft portion.
In Example 18, the medical catheter of Example 16, further comprising one or more steering wire lumens within the articulation structure and configured to receive respective steering elements for applying the deflection force, wherein the one or more steering wire lumens are each circumferentially offset from the first and second reinforcing members by about 90 degrees and are positioned to align with the reinforcing sleeves.
In Example 19, the medical catheter of Example 18, wherein the reinforcing sleeves provide lateral support for the one or more steering elements extending through the proximal shaft portion.
In Example 20, the medical catheter of Example 16, wherein each of the reinforcing sleeves is in the form of a helically coiled ribbon.
In Example 21, the medical catheter of Example 20, wherein the anchor ring includes passageways for the one or more steering elements extending through the proximal shaft portion.
In Example 22, the medical catheter of Example 16, wherein the anchor ring is positioned adjacent to the transition portion of the shaft.
In Example 23, the medical catheter of Example 22, wherein the transition portion includes an area of overlapped reinforcing braid sections comprising the first length of the proximal reinforcing braid and the second length of distal reinforcing braid.
In Example 24, the medical catheter of Example 23, further comprising a mechanical connection between the first length of the proximal reinforcing braid and the second length of the distal reinforcing braid.
In Example 25, the medical catheter of Example 24, wherein the mechanical connection further connects the anchor ring to the first length of the proximal reinforcing braid and the second length of the distal reinforcing braid.
In Example 26, the medical catheter of Example 25, wherein the transition portion includes polymeric material reflowed over and encapsulating the mechanically connected first length of the proximal reinforcing braid, second length of the distal reinforcing braid, and anchor ring.
In Example 27, the medical catheter of Example 26, wherein the mechanical connection is formed by a welding process, soldering process, adhesive process, polymer reflow process, or combination thereof.
In Example 28, a shaft for a steerable medical catheter, the shaft comprising a tubular member having a proximal portion, a distal portion, and a transition portion between the proximal and distal portions of the shaft. The proximal portion includes a proximal shaft component having a proximal reinforcing braid and a proximal polymeric jacket disposed over the proximal reinforcing braid, wherein a first length of the proximal reinforcing braid extends distally of the proximal polymeric jacket. The distal portion includes from a distal shaft component having a distal reinforcing braid and a distal polymeric jacket disposed over the distal reinforcing braid, wherein a second length of the distal reinforcing braid extends proximally of the distal polymeric jacket and is mechanically connected to the first length of the proximal reinforcing braid. The transition portion of the shaft comprises the first length of the proximal braid member, the second length of the distal braid member, and a transition portion jacket disposed over the first length of the proximal braid member and the second length of the distal braid member.
In Example 29, the shaft of Example 28, wherein the proximal portion comprises a reinforcing assembly disposed within the proximal shaft component. The reinforcing assembly comprises a pair of diametrically opposed helical reinforcing sleeves, wherein the reinforcing sleeves establish reinforced passageways configured to provide lateral support for one or more steering elements, and an anchor ring attached to a distal end of each of the reinforcing sleeves, the anchor ring comprising a ring structure and includes passageways for the one or more steering elements, and wherein the anchor ring is mechanically connected to the proximal shaft component and the distal shaft component in the transition portion.
In Example 30, the shaft of Example 29, wherein the distal portion comprises an articulation structure disposed within the distal shaft component. The articulation structure is configured to enable deflection of the distal shaft portion along a curved plane and comprises a plurality of longitudinally-arranged tubular segments and a plurality of connecting segments connecting adjacent tubular segments, a pair of diametrically opposed steering wire lumens extending through the tubular segments and aligned with the reinforcing sleeves, and first and second reinforcing members extending through the tubular segments and the connecting segments.
In Example 31, the shaft of Example 30, wherein the anchor ring is mechanically connected to the first length of the proximal reinforcing braid and the second length of the distal reinforcing braid.
In Example 32, the shaft of Example 31, wherein the anchor ring includes a groove defined in its outer circumference, and wherein the anchor ring is mechanically connected to the first length of the proximal reinforcing braid and the second length of the distal reinforcing braid by disposing an adhesive material over the first length of the proximal reinforcing braid and the second length of the distal reinforcing braid and into the groove.
In Example 33, the shaft of Example 31, wherein the transition portion includes polymeric material reflowed over and encapsulating the mechanically connected first length of the proximal reinforcing braid, second length of the distal reinforcing braid, and anchor ring.
In Example 34, a method of producing an integrated medical catheter comprising providing an elongated proximal shaft portion including a proximal reinforcing braid disposed within a proximal polymeric jacket, wherein a first length of the proximal reinforcing braid extends from of the proximal polymeric jacket, providing an elongated distal shaft portion including a proximal reinforcing braid disposed within a distal polymeric jacket, wherein a second length of the proximal reinforcing braid extends from of the distal polymeric jacket, positioning the first length of the proximal reinforcing braid adjacent to the second length of the distal reinforcing braid, providing a reinforcing assembly that comprises of a pair of diametrically opposed helical reinforcing sleeves, and an anchor ring attached to the distal end of the reinforcing sleeves, positioning the reinforcing assembly within the proximal shaft portion such that the anchor ring is positioned adjacent the first length of the proximal reinforcing braid and the second length of the distal reinforcing braid, and mechanically connecting the first length of the proximal reinforcing braid and, the second length of the distal reinforcing braid, and the anchor ring to integrate the proximal and distal shaft portions into an elongated catheter shaft having a transition portion at a junction of the proximal and distal shaft portions.
In Example 35, the method of Example 34, further comprising reflowing polymeric material over the first length of the proximal reinforcing braid and the second length of the distal reinforcing at the transition portion.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an illustration of an example medical catheter, consistent with various aspects of the present disclosure.

FIG. 2A is an illustration of the medical catheter, shown in FIG. 1, as deflected in a first direction, consistent with various aspects of the present disclosure.

FIG. 2B is an illustration of the medical catheter, shown in FIG. 1, as deflected in a second direction, consistent with various aspects of the present disclosure.

FIG. 3 is a detailed illustration of a portion of a side-view illustration of the medical catheter, consistent with various aspects of the present disclosure.

FIG. 4A is an illustration of a reinforcing assembly enclosed in the medical catheter with a grooved anchor ring attachment, consistent with various aspects of the present disclosure.

FIG. 4B is an illustration of a reinforcing assembly enclosed in the medical catheter with a smooth, non-grooved anchor ring attachment, consistent with various aspects of the present disclosure.

FIG. 5 is a top-view illustration of a reinforcing assembly of the medical catheter, consistent with various aspects of the present disclosure.

FIG. 6A is an illustration of an articulation structure and reinforcing assembly with a grooved anchor ring enclosed in the medical catheter, consistent with various aspects of the present disclosure.

FIG. 6B is an illustration of an articulation structure and reinforcing assembly with a non-grooved anchor ring enclosed in the medical catheter, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

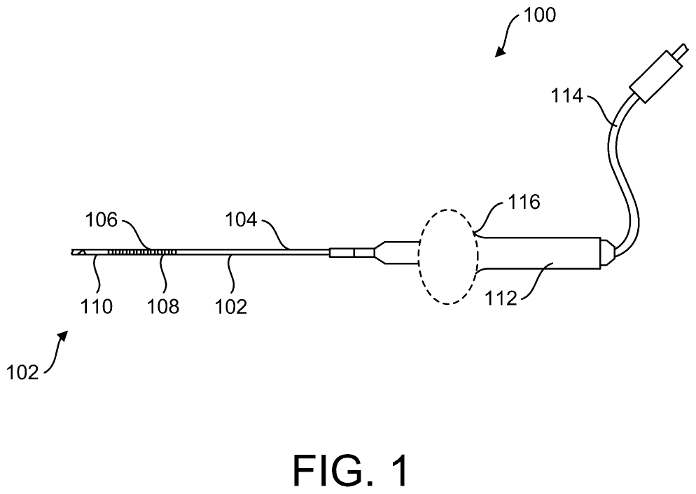

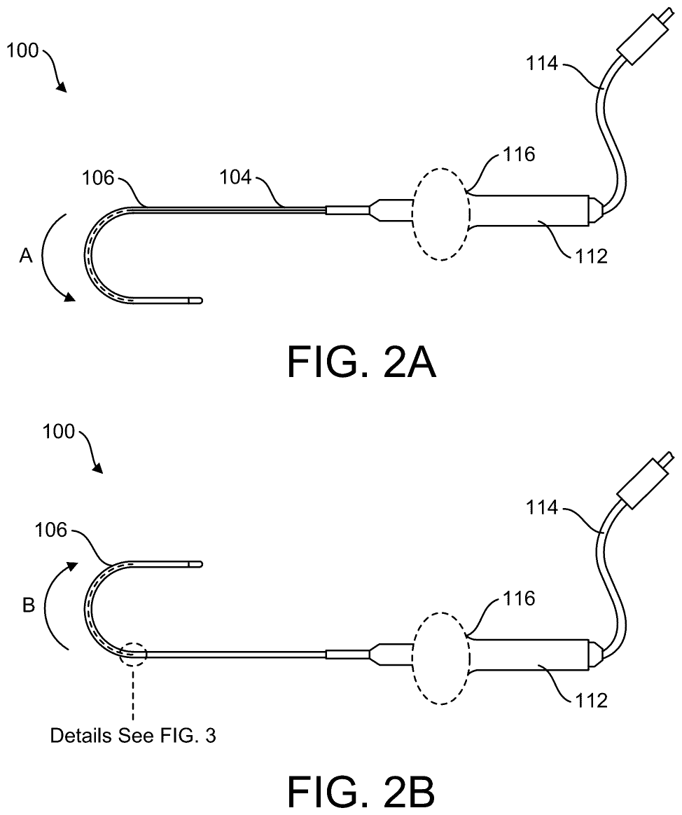

FIG. 1, FIG. 2A and FIG. 2B are illustrations of an example medical catheter 100, consistent with various aspects of the present disclosure. As shown in FIG. 1, the medical catheter 100 may be a steerable medical catheter 100. In certain instances, the medical catheter 100 is steerable in one direction (e.g., direction A as shown in FIG. 2A) or in another direction (e.g., direction B as shown in FIG. 2B). The medical catheter 100 generally includes a tubular shaft 102 having a proximal portion 104 and a distal portion 106 that is sized and configured for placement and manipulation within in a target area of a patient. The distal portion 106 may be steerable. As shown, the distal portion 106 further includes a deflection region 108 and a distal end 110.

In certain instances, the tubular shaft 102 extends from a distal portion of a handle 112. An electrical cable or other suitable connector 114 extending from a proximal end of the handle 112 may be coupled to a source of energy or other equipment (not shown in FIG. 1) for transmitting one or more ablation signals. A steering actuator 116, such as a rotatable knob or plunger that may be arranged at the handle 112, may be manipulated by a physician to deflect or position the steerable distal portion 106 of the tubular shaft 102.

As shown in FIGS. 2A and 2B, the medical catheter 100 is of the deflectable or steerable type, such that during use, the deflection region 108 can be deflected or curved by a user to facilitate locating the distal end 110 at a desired target location within the heart. In embodiments, deflection of the deflection region 108 can be accomplished by manipulation of the steering actuator 118, which is operatively connected to steering elements (e.g., wires, tendons, ribbons, and the like) extending within and attached (directly or indirectly) to the shaft 102 at a location within the distal portion 106. The particular mode and structure for deflecting the deflection region 108 is not critical to the present disclosure, and so any technique, whether now known or later developed, can be employed within the scope of the present disclosure.

In use, deflecting or curving the distal portion 106 may impart a torsional force that could torque or twist the distal portion 106 away from or out of the target location. The deflection region 108 may torque out of plane from the plane in which the distal portion 106 was arranged prior to deflection due to the tension on the curvature of the medical catheter 100 within vasculature.

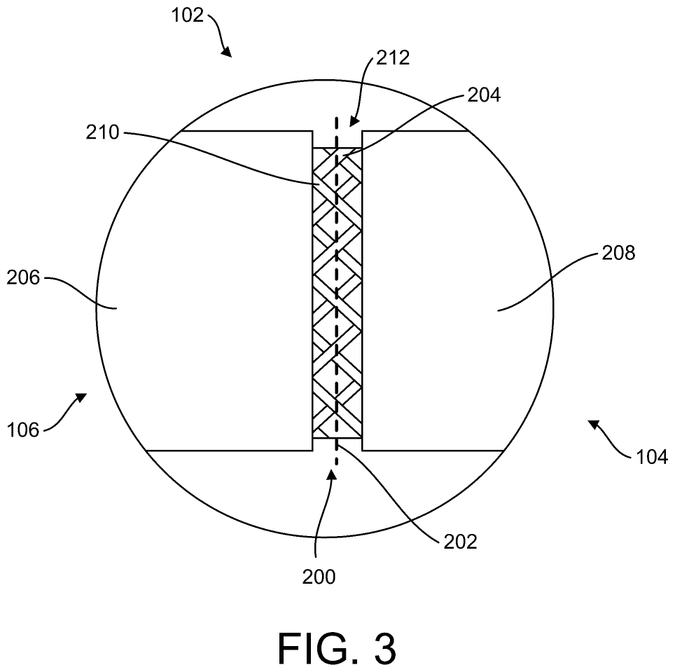

FIG. 3 is an enlarged detail view of the medical catheter 100 illustrating a mechanical connection 202 of the proximal shaft 104 and the distal shaft 106 creating a transition portion 200. As shown, shaft 102 includes proximal polymeric jacket 208 encapsulating the proximal portion of the shaft 104, and a distal polymeric jacket 206 encapsulating the distal portion of the shaft 106. In certain instances, the proximal portion 104 and the distal portion 106 may be constructed of different materials to impart specific performance characteristics. For example, the proximal polymeric jacket 208 and the distal polymeric jacket 206 may be of a braided construction, wherein the proximal polymeric jacket 208 and the distal polymeric jacket 206 each having a polymer material reinforced with a distinct reinforcing braid formed of a plurality of interwoven wires that are woven, knitted, entwined or otherwise interlaced together.

As shown in FIG. 3, the shaft 102 includes a proximal polymeric jacket 208 encapsulating the proximal portion 104, and a distal polymeric jacket 206 encapsulating the distal portion 106. To integrate the proximal portion 104 and the distal portion 106 into a unified device, a user may strategically remove the polymeric jacket 360 degree around the adjoining ends of the proximal and distal shaft portions, through a removal process (e.g. ablation process or other removal process), to expose underlying reinforcing braids. The proximal polymeric jacket 208 may be removed from a terminal segment of the proximal portion 104, revealing an extending length of proximal reinforcing braid 204. Similarly, the distal polymeric jacket 206 may be removed from the adjoining end of the distal portion 106, exposing a length of distal reinforcing braid 210. The exposed braids 204, 210 originate from independent spools within their respective shaft jackets. The exposed proximal reinforcing braid 204 and distal reinforcing braid 206 may be mechanically connected to create the transition portion 200 of the shaft 102 through the mechanical connection 202 wherein the transition portion 200 may create a braided gap 212 between the proximal portion 104 and the distal portion 106. In various embodiments, the braided gap 212 may have a length of about 0.030 inches to about 0.300 inches. The mechanical connection 202 may provide several advantages in allowing for a gradual transition of mechanical properties, such as flexibility and torque response, between the disparate proximal and distal shaft materials. The mechanical connection allows the integrated medical catheter 100 maneuverability and overall performance of the catheter. Additionally, in some embodiments, a user may make modifications after the mechanical connection is accomplished, such as further material encapsulation, to fine-tune catheter performance.

In some embodiments, the mechanical connection 202 may be done through welding process, soldering process, adhesive process, polymer reflow process, or other processes that may enhance the connection between the proximal reinforcing braid 204 and the distal reinforcing braid 210.

In some embodiment, polymeric material may be reflowed over and around the mechanically connected reinforcing braids 204, 210 to seal and encapsulate the mechanically connected components into a polymeric jacket, wherein the reflowed polymeric material is from the existing polymeric jacket or a polymeric material same composition as the existing polymeric jacket or a compatible material.

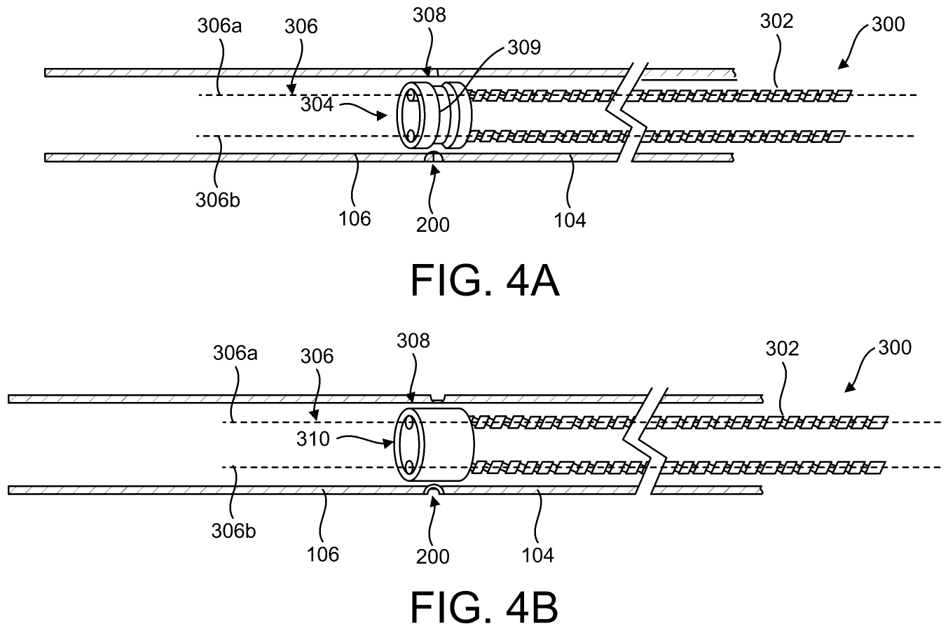

Referring now to FIG. 4A and FIG. 4B, a reinforcing assembly 300 is introduced within the medical catheter 100. The reinforcing assembly 300, is a component within the medical catheter that facilitates steering control and maneuverability with the integration of reinforcing sleeves 302 which may act as a reinforced steering wire guidance system within the catheter shaft, allowing for controlled deflection of the distal portion 106 while providing support and stability to the proximal portion 104. The reinforcing assembly 300 is positioned within the proximal portion 104 of the medical catheter shaft 102 and extends into the distal portion 106 and comprises reinforcing sleeves 302 and an anchor ring (304 or 310), wherein the anchor ring may be composed from a metallic material or of a similar alloy. The reinforcing sleeves 302 may comprise a pair of diametrically opposed helical coils that extends longitudinally through the proximal portion 104 of the shaft 102. Further, the reinforcing sleeves 302 create dedicated passageways for one or more steering elements (e.g., wires, tendons, ribbons, and the like) 306, wherein the one or more steering element may comprise a first steering element 306a and a second steering element 306b. The one or more steering elements 306 are located in diametrically opposite positions and are operatively connected to the steering actuator 118 (FIG. 1) to facilitate the deflection of at least the deflection region 108 of the tubular shaft 102 as known in the conventional manner. Additionally, the reinforcing sleeves 302 may provide radial and lateral support and guidance for the one or more steering elements 306 by encasing them within the helical coils. The protective structure may help minimize the impact of any compressive or any other forces that may occur during catheter manipulation allowing the steering elements to maintain their integrity and functionally for consistent and precise deflection of the deflection region 108.

Further, at a distal end of the reinforcing assembly 308, the reinforcing sleeves 302 are securely attached to the anchor ring (304 or 310) positioned near or at the transition portion 200 serving as an anchoring point, wherein the anchor ring (304 or 310) includes passageways for the one or more steering elements 306 extending from the proximal region to the distal region. In an example embodiment, the anchor ring is illustrated with a grooved anchor ring 304 (FIG. 4A). In another embodiment the anchor ring is shown with a smooth, non-grooved anchor ring 310 (FIG. 4B). The grooved anchor ring 304 of FIG. 4A features a circumferential groove 309, wherein the circumferential groove may accommodate an adhesive. The circumferential groove 309 allows an adhesive to fill the space, creating a connection between the anchor ring 304 and the transition portion 200. The adhesive may flow through the braided gap 212 and flow into the groove, filling the voids and forming a strong bond that prevents slippage and maintains the integrity of the reinforcing assembly 300. The secure attachment allows for a seamless transfer of steering forces from the proximal portion 104 to the distal portion 106, allowing control and maneuverability of the medical catheter 100.

In some embodiment, the anchor ring 304 may be mechanically connected through welding process, soldering process, adhesive process, polymer reflow process, or other processes that may enhance the connection between the anchor ring 304 and the transition portion 200.

In some embodiment, polymeric material may be reflowed over and around the mechanically connected reinforcing braids 204, 210 and the anchor ring 304 to seal and encapsulate the mechanically connected components into a polymeric jacket.

In contrast, FIG. 4B illustrates the smooth, non-grooved anchor ring 310. While lacking the circumferential groove, the smooth anchor ring 310 may still be effectively attached to the transition portion 200 using suitable mechanical means of connection. These means of mechanical connection may be done through welding process, soldering process, adhesive process, polymer reflow process, or other processes that may allow the connection between the anchor ring 310 and the transition portion 200. The choice between a grooved anchor ring 304 and the smooth anchor ring 310 may depend on various factors, including the desired level of attachment strength, manufacturing feasibility, and specific requirements of the medical catheter application.

In some embodiment, polymeric material may be reflowed over and around the mechanically connected reinforcing braids 204, 210 and the anchor ring 310 to seal and encapsulate the mechanically connected components into a polymeric jacket.

Regardless of the anchor ring design, the strategic positioning and mechanical attachment of the anchor ring (304 or 310) to the transition portion 200 allows transfer of steering forces from the proximal portion 104 to the distal portion 106. The mechanical connection may help with overall control and maneuverability of the medical catheter 100 enabling precise navigation through challenging anatomical structures.

In some embodiments, the reinforcing assembly 300 may be adapted to suit specific catheter designs and applications. Variations in the number, configuration, and/or material composition of the reinforcing sleeves 302 and anchor rings (304 or 310) may be employed to optimize performance characteristics. For example, the helical coils of the reinforcing sleeves may be designed with different diameters, or the anchor rings may be constructed of various materials to achieve the desired strength, and flexibility.

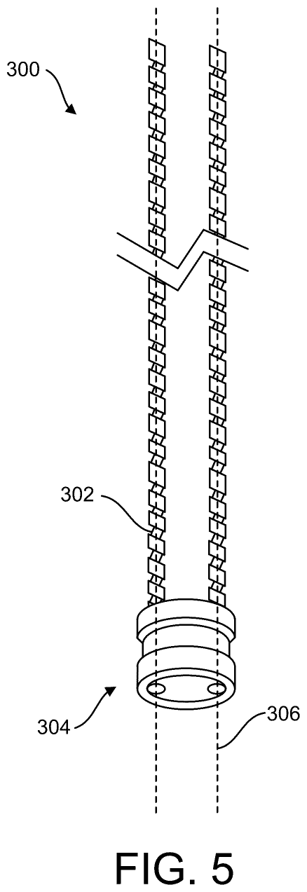

FIG. 5A is a top view illustration of the reinforcing assembly 300 as previously described in FIG. 4A and FIG. 4B. As shown, the reinforcing assembly includes the reinforcing sleeves 302 wherein the reinforcing sleeves 302 create dedicated passageways 320, 322 for the one or more steering elements 306. The one or more steering elements 306 may be located in diametrically opposite positions and are operatively connected to the steering actuator 118 (FIG. 1) to facilitate the deflection of at least the deflection region 108 of the tubular shaft 102 as known in the conventional manner.

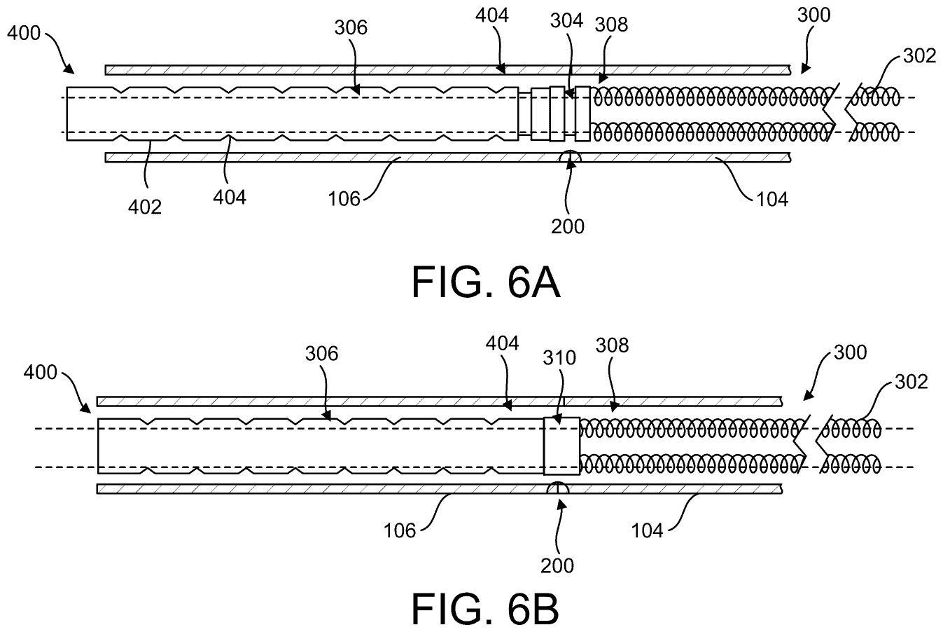

FIG. 6A and FIG. 6B introduces the articulation structure 400 that may be positioned within the distal portion of the medical catheter shaft 102 and facilitates predictable, highly planar deflection of the deflection region 108 by resisting torsional forces on the shaft 102 that would otherwise tend to cause the deflection region 108 to deflect. The articulation structure 400 may comprise series of interconnected plurality of longitudinally-arranged articulation elements that work together to achieve the desired deflection characteristics. These articulation elements may comprise a plurality of longitudinally arranged tubular segments, a plurality of connecting segments, plurality of articulation links, a plurality of joints, a plurality of reinforcing members, or combinations thereof. In the illustrated embodiment, the articulation structure includes a plurality of tubular segments 402 and a plurality of connecting segments 404 connecting adjacent tubular segments 402. The articulation structure 400 may include one or more reinforcing members (not shown) embedded within the articulation elements and extending through the articulation structure 400 wherein the reinforcing members may maintain a curved shape of the deflection region 108 during deflection and maintain repeatability of achieving the curved shape of the deflection region 108 during deflection. In an example embodiment the one or more reinforcing members may comprise a helically wound flat ribbon wire or stranded wire cables or of similar elements. The articulation structure 400 may further comprise one or more steering wire lumens arranged in parallel to the passageways 320, 322 of the reinforcing assembly 300 wherein the steering wire lumens may be configured to receive one or more steering elements 306 that are operatively connected to the steering actuator 118 (FIG. 1) to facilitate the deflection of at least the deflection region 108 of the tubular shaft 102 as known in the conventional manner. The one or more steering wire lumens may also be circumferentially offset from the one or more reinforcing members by about 90 degrees. The specific design and arrangement of these elements can vary depending on the desired deflection profile and the intended application of the medical catheter 100. As shown, the articulation structure 400 may be positioned in close proximity to the reinforcing assembly 300 wherein the distal end of the reinforcing assembly 308 is touching or near the proximal end of the articulation structure 404. This placement allows transfer of steering forces from the reinforcing assembly 300 to the articulation structure 400, allowing smooth and controlled deflection of the distal portion of the medical catheter shaft 102. In response to a deflection force, the articulation structure 400 may bend in a direction A (FIG. 2A). In certain embodiments, the articulation structure 400 may bend in another direction B (FIG. 2B), opposite to that of direction A. Whether the articulation structure 400 is unidirectional or bidirectional is not of critical importance to the current invention.

The articulation structure 400 being arranged within the deflection region 108 of the tubular shaft 102 operates to stabilize at least the distal portion 106 of the tubular shaft 102. In other instances, the articulation structure 400 may extend along a length of the tubular shaft 102 or extend into an intermediate section between the proximal portion 104 and the distal portion 106.

The articulation structure 400 embedded within the deflection region 108 of the tubular shaft 102 may be caused to assume a curved shape when the tubular shaft 102 is arranged within a patient's vasculature. The ability to selectively curve the deflection region 108 can facilitate accurate and effective delivery therapy (e.g., ablation) or map the surface of the heart tissue (e.g., identify the locations of heart tissue that are a source of the arrhythmias).

In some embodiments, the articulation structure 400 may include a center lumen wherein the center lumen may include components such as one or more wires for ablation electrodes arranged along the shaft 102, navigational components, a temperature sensor (e.g., thermocouple), a force sensor, radio-frequency circuitry and/or wires, and a cooling lumen. The center lumen may be a working channel through which one or more devices may be passed.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more. ” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical catheter comprising:
a handle configured for manipulation by a user;
an elongate proximal shaft portion extending from the handle and comprising a proximal reinforcing braid disposed within a proximal polymeric jacket, wherein a first length of the proximal reinforcing braid extends distally beyond the proximal polymeric jacket;
an elongate distal shaft portion comprising a distal reinforcing braid disposed within a distal polymeric jacket, wherein a second length of the distal reinforcing braid extends proximally beyond the distal polymeric jacket, wherein the first length of proximal reinforcing braid and the second length of distal reinforcing braid are mechanically connected to form an integrated transition portion joining the proximal and distal shaft portions;
a reinforcing assembly extending proximally from the transition portion within the proximal shaft portion, wherein the reinforcing assembly comprises a pair of diametrically opposed helical reinforcing sleeves, and an anchor ring attached to the distal end of the reinforcing sleeves, wherein the reinforcing sleeves establish passageways for one or more steering elements extending through the proximal shaft portion; and
an articulation structure extending distally from the transition portion, wherein the articulation structure comprises a plurality of longitudinally-arranged articulation elements, wherein the articulation elements comprise a plurality of tubular segments and a plurality of connecting segments connecting adjacent tubular segments, the articulation structure configured to enable deflection of the distal shaft portion along a curved plane.

2. The medical catheter of claim 1, wherein the articulation structure is configured to assume a curved shape when a deflection force is applied to the distal shaft portion.

3. The medical catheter of claim 1, further comprising one or more steering wire lumens within the articulation structure and configured to receive respective steering elements for applying the deflection force, wherein the one or more steering wire lumens are each circumferentially offset from the connecting segments by about 90 degrees and are positioned to align with the reinforcing sleeves.

4. The medical catheter of claim 3, wherein the reinforcing sleeves provide lateral support for the one or more steering elements extending through the proximal shaft portion.

5. The medical catheter of claim 1, wherein each of the reinforcing sleeves is in the form of a helically coiled ribbon.

6. The medical catheter of claim 5, wherein the anchor ring includes passageways for the one or more steering elements extending through the proximal shaft portion.

7. The medical catheter of claim 1, wherein the anchor ring is positioned adjacent to the transition portion of the shaft.

8. The medical catheter of claim 7, wherein the transition portion includes an area of overlapped reinforcing braid sections comprising the first length of the proximal reinforcing braid and the second length of distal reinforcing braid.

9. The medical catheter of claim 8, further comprising a mechanical connection between the first length of the proximal reinforcing braid and the second length of the distal reinforcing braid.

10. The medical catheter of claim 9, wherein the mechanical connection further connects the anchor ring to the first length of the proximal reinforcing braid and the second length of the distal reinforcing braid.

11. The medical catheter of claim 10, wherein the transition portion includes polymeric material reflowed over and encapsulating the mechanically connected first length of the proximal reinforcing braid, second length of the distal reinforcing braid, and anchor ring.

12. The medical catheter of claim 11, wherein the mechanical connection is formed by a welding process, soldering process, adhesive process, polymer reflow process, or combination thereof.

13. A shaft for a steerable medical catheter, the shaft comprising:
a tubular member having a proximal portion, a distal portion, and a transition portion between the proximal and distal portions of the shaft, wherein:
the proximal portion includes a proximal shaft component having a proximal reinforcing braid and a proximal polymeric jacket disposed over the proximal reinforcing braid, wherein a first length of the proximal reinforcing braid extends distally of the proximal polymeric jacket, and
the distal portion includes from a distal shaft component having a distal reinforcing braid and a distal polymeric jacket disposed over the distal reinforcing braid, wherein a second length of the distal reinforcing braid extends proximally of the distal polymeric jacket and is mechanically connected to the first length of the proximal reinforcing braid, and
wherein the transition portion of the shaft comprises the first length of the proximal braid member, the second length of the distal braid member, and a transition portion jacket disposed over the first length of the proximal braid member and the second length of the distal braid member.

14. The shaft of claim 13, wherein the proximal portion comprises a reinforcing assembly disposed within the proximal shaft component, the reinforcing assembly comprising:
a pair of diametrically opposed helical reinforcing sleeves, wherein the reinforcing sleeves establish reinforced passageways configured to provide lateral support for one or more steering elements; and
an anchor ring attached to a distal end of each of the reinforcing sleeves, the anchor ring comprising a ring structure and includes passageways for the one or more steering elements, and wherein the anchor ring is mechanically connected to the proximal shaft component and the distal shaft component in the transition portion.

15. The shaft of claim 14, wherein the distal portion comprises an articulation structure disposed within the distal shaft component, the articulation structure configured to enable deflection of the distal shaft portion along a curved plane and comprising:
a plurality of longitudinally-arranged tubular segments and a plurality of connecting segments connecting adjacent tubular segments; and
a pair of diametrically opposed steering wire lumens extending through the tubular segments and aligned with the reinforcing sleeves.

16. The shaft of claim 15, wherein the anchor ring is mechanically connected to the first length of the proximal reinforcing braid and the second length of the distal reinforcing braid.

17. The shaft of claim 16, wherein the anchor ring includes a groove defined in its outer circumference, and wherein the anchor ring is mechanically connected to the first length of the proximal reinforcing braid and the second length of the distal reinforcing braid by disposing an adhesive material over the first length of the proximal reinforcing braid and the second length of the distal reinforcing braid and into the groove.

18. The shaft of claim 16, wherein the transition portion includes polymeric material reflowed over and encapsulating the mechanically connected first length of the proximal reinforcing braid, second length of the distal reinforcing braid, and anchor ring.

19. A method of producing an integrated medical catheter comprising:
providing an elongated proximal shaft portion including a proximal reinforcing braid disposed within a proximal polymeric jacket, wherein a first length of the proximal reinforcing braid extends from of the proximal polymeric jacket;
providing an elongated distal shaft portion including a proximal reinforcing braid disposed within a distal polymeric jacket, wherein a second length of the proximal reinforcing braid extends from of the distal polymeric jacket;
positioning the first length of the proximal reinforcing braid adjacent to the second length of the distal reinforcing braid;
providing a reinforcing assembly that comprises of a pair of diametrically opposed helical reinforcing sleeves, and an anchor ring attached to the distal end of the reinforcing sleeves;
positioning the reinforcing assembly within the proximal shaft portion such that the anchor ring is positioned adjacent the first length of the proximal reinforcing braid and the second length of the distal reinforcing braid; and
mechanically connecting the first length of the proximal reinforcing braid and, the second length of the distal reinforcing braid, and the anchor ring to integrate the proximal and distal shaft portions into an elongated catheter shaft having a transition portion at a junction of the proximal and distal shaft portions.

20. The method of claim 19, further comprising reflowing polymeric material over the first length of the proximal reinforcing braid and the second length of the distal reinforcing at the transition portion.
